(19) United States
(12) Patent Application Publication
(10) Pub. No.: US 2022/0264971 A1
(43) Pub. Date: Aug. 25, 2022

(54) Therapeutic Treatment Garment

(71) Applicant: Norma DeLara, Westbrook, CT (US)

(72) Inventor: Norma DeLara, Westbrook, CT (US)

(21) Appl. No.: 17/183,469

(22) Filed: Feb. 24, 2021

(51) Int. Cl. A41D 13/12; A41D 27/20

(52) U.S. Cl. CPC A41D 13/1245; A41D 27/20; A41D 2400/32; A41D 2500/20; A41D 2500/30; A41D 2300/32; A41D 2300/324; A41D 2300/322; A41D 2300/326

(57) ABSTRACT

The present invention relates to a unique therapeutic garment that wraps around the torso of a patient with a chest related ailment or recovering from a chest related procedure such as, without limitation, a mastectomy, lumpectomy, or the like. The therapeutic garment comprises a web, band or tube-like post-surgical wrap having two opposite ends along the length of the garment, which are secured together over the torso of the user. The therapeutic garment further comprises two pockets that are attached to, or integrally formed with, the wrap garment, such that one pocket covers the left breast of the wearer and the other pocket covers the right breast. Each of the pockets has an opening therein for receipt of a hot pack, cold pack and/or therapeutic device to combat swelling, pain and the like.

### FIELD OF THE INVENTION

The present invention relates generally to the field of therapeutic garments. More specifically, the present invention relates to a therapeutic compression garment that wraps around the chest of a patient diagnosed with a chest related medical condition or recovering from a chest related surgical procedure, such as a mastectomy. The therapeutic garment has two slits or openings along its front portion for receipt of hot or cold packs, or other therapeutic devices. The hot/cold packs and therapeutic devices help to counteract the wearer's pain, swelling, muscle spasms, and/or the general discomfort experienced by a user post-mastectomy or other procedure, and therefore helps reduce the user's dependency on pain medication. Accordingly, the present specification makes specific reference thereto. However, it is to be appreciated that aspects of the present invention are also equally usable with other like applications, devices and methods of manufacture.

### BACKGROUND OF THE INVENTION

By way of background, women and men diagnosed with breast cancer oftentimes undergo various treatments including breast surgeries, chemotherapy, radiation and the like in an effort to treat their disease. While breast cancer in males occurs less often than in females, it does arise for older males. Breast cancer is a form of cancer that develops within the breast tissue. Signs of breast cancer may include a lump in the breast, a change in the shape of the breast, dimpling of the skin, fluid coming from the nipple, or a red scaly patch of skin on the breast. The diagnosis of breast cancer is oftentimes confirmed by taking a biopsy of the concerning lump. Once the diagnosis is made, further tests are done to determine which treatment will be most effective. The surgical options include a lumpectomy or partial mastectomy, a single mastectomy or a double mastectomy, followed by radiation therapy in some critical and invasive cases. Additionally, other breast related issues may require a patient to undergo surgery, which may frequently cause post-op inflammation. Unfortunately, while this surgery is a part of the treatment regiment, it oftentimes results in pain, swelling, and discomfort in the chest area of the patient.

Further, patients undergoing breast augmentation may also suffer from pain, discomfort and swelling post-surgery. Medical professionals treating such patients oftentimes use pain medication to combat these symptoms and reduce the patient's pain. However, prolonged and excessive use of pain medication may have side effects and otherwise affect the patient's body and its functioning, particularly in the case of opioids which can be addictive and cause longer term health issues.

Additionally, while some patients may take pain medications to combat these symptoms, many people prefer natural pain control methods, such as applying hot or cold packs or other home remedies or devices to the affected area. For proper treatment, the hot or cold pack is typically kept over the affected chest area for approximately 10-20 minutes of every hour. Unfortunately, it is difficult to keep the hot or cold packs in the correct position without proper support as the user must either continue to hold the same or limit his or her movement so the hot/cold packs are not displaced. Further, most heating pads that are used to soothe patients do not fit or conform well around the breast, and therefore provide only partial relief Also, heating pads limit movement of the patient due to the length of the electrical cord used to provide power to the heating pad. It is also difficult for the patient to hold the hot or cold packs while moving around or performing different tasks.

Other treatments, such as compression therapy, can also be effective for treating the chest area of a patient post-surgical procedure. During compression therapy, a compression sleeve or other garment providing a compressive force is used by the patient to apply pressure to the affected area to increase blood flow to the affected area and promote healing. In addition, compression therapy has also been used to treat patients suffering from anxiety, such as those created by psychological or neurological disorders. Unfortunately, it is oftentimes difficult to use both compression therapy and the hot or cold packs at the same time.

Therefore, there exists a long felt need in the art for an effective treatment to combat post-operative inflammation, swelling in the chest area, pain, anxiety and the like. There is also a long felt need in the art for a therapeutic treatment garment that enables a wearer to apply heat or cold to an affected area in a hands free and secure manner, thereby enabling the wearer to engage in other tasks while receiving treatment. Additionally, there is a long felt need in the art for a therapeutic treatment garment that provides compression therapy to the affected area both during the application of the heat/cold and during such times that the same is not being applied. Moreover, there is a long felt need in the art for a therapeutic treatment garment that comprises waterproof pockets for securing and applying an ice pack to an affected area without dampening the skin of the wearer. Finally, there is a long felt need in the art for a therapeutic treatment garment that is relatively inexpensive to manufacture, and safe and easy to use.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a therapeutic treatment garment to be worn around, for example, the chest of a post-operative mastectomy or lumpectomy patient to provide compression, heat/coolness and pain relief. The therapeutic treatment garment is comprised of a web, band or tube-like post-surgical wrap with two opposite ends along the length of the garment, wherein one end is comprised of, for example, a hook fastener, and the other end is comprised of a loop fastener. The hook and loop fasteners are used to secure the opposite ends of the therapeutic treatment garment together in the front or back of the patient wearing the garment. A top end of the garment is comfortably secured around and above the chest region of the patient, and the bottom edge of the garment is open like a raincoat or a long coat. The therapeutic treatment garment is further comprised of two water resistant pockets covering a right and a left chest area, wherein each of the pockets has an open slit for inserting a hot or a cold pack, a compression device or other therapeutic aid into each pocket to reduce pain and swelling post-operatively. The slits may have a thin magnetic strip for opening and closing the pockets for inserting the hot or cold pack, compression device or other therapeutic aid. The pockets are large and deep enough to cover the whole chest area, but may also be configured to cover only a portion of the chest area as needed by the individual.

The subject matter disclosed and claimed herein, in a further embodiment thereof, comprises a therapeutic garment to be worn around the chest of a mastectomy or lumpectomy patient to provide post-operative compression and/or pain relief. The therapeutic garment comprises a web, band or tube-like post-surgical wrap having two opposite ends along the length of the garment. One end has a hook fastener, and the opposite end has a loop fastener. The hook and loop fasteners are used to secure the opposite ends together in front of the patient wearing the garment. A top end of the garment is comfortably secured around, and may include an elastic strap or band to assist in holding the garment in position (above the chest region of the patient) and the bottom edge of the garment is open. The wrap garment has two slits on the top half of the garment disposed at equal distance from the opposite ends such that when the wrap garment is worn, the slits come in front and are covering the chest/breast area. Alternatively, the slits may be positioned on the side of the pockets. The slits lead to water resistant pockets into which a hot or cold pack is inserted to reduce pain and swelling. Other compression or therapeutic devices may also be inserted in the pockets. The slits have a closure mechanism for opening and closing the slits to, for example, insert a hot or cold pack or other therapeutic device therein. The pockets are large and deep enough to cover the whole chest area, and the wrap garment is comfortable and gives firm support that is not too tight. The wrap garment can be worn daily, and can also be worn when the patient is doing any form of exercise, physical therapy, yoga or other activity. The wrap garment of the present invention is used by the patients who have recently undergone a lumpectomy, mastectomy, double mastectomy, breast replacement, breast augmentation or other chest related surgery to control post-operative inflammation, pain, swelling and/or discomfort in the chest area.

In a further embodiment of the present invention, a chest wrap that offers compression and pain relief to patients having undergone some sort of chest surgery is disclosed. The chest wrap comprises a therapeutic garment having two front water-resistant pockets with slits or openings therein, wherein the pockets cover the chest area of the patient. Each pocket has an interior area that is accessible through the slit of the corresponding pocket. The interior area is used to hold a hot or cold pack, wherein the hot or cold pack provides relief from pain, swelling, muscle spasms and the like. The slits may have a closure mechanism such as, but not limited, to a zipper, magnet, snap, clasp, button or other suitable fastener to access the interior area of the pockets.

In a further embodiment of the present invention, a therapeutic garment to reduce pain and swelling after a chest surgery and to conform securely to a patient's chest and torso is disclosed. The therapeutic garment comprises a web, band or tube-like surgical wrap fabricated of a stretchable material, wherein the wrap is comprised of a top edge, a bottom edge, a first longitudinal edge, and a second longitudinal edge. The first longitudinal edge has hook fasteners along the edge length, and the second longitudinal edge has loop fasteners along the edge length, wherein the hook and loop fasteners are used to secure the opposite longitudinal edges together in front of the patient when the wrap is worn. Two waterproof pockets are stitched or positioned on an exterior surface of the wrap, wherein each pocket is closed and stitched on three edges with a slit having a magnetic or other closure such that a hot or cold pack or other therapeutic aid or device is inserted into each pocket and removed from each pocket to provide relief from pain and discomfort to the user. The magnetic closure system of the slit may be made up of a single magnetic strip or a plurality of magnetic units, and can be used to close the pocket and secure any items therein, such as hot or cold packs or therapeutic devices. The pockets are stitched, positioned or secured in such a way as to provide maximum coverage of the therapeutic device to the affected area of the chest.

In yet another embodiment of the present invention, a method for fabricating a therapeutic garment to be worn by a patient to get relief from pain, swelling and discomfort in the chest is disclosed and comprises the step of initially taking a web, band or tube like rectangular wrap garment of a material such as Lycra, cotton or similar material having an elastic ability to distribute pressure evenly across the chest area. Next, a fastening mechanism, such as Velcro, snaps, buttons or hooks, is attached on the longitudinal edges of the rectangular wrap garment. First and second pockets using the same material are then applied on the outer layer of the wrap garment by, for example, stitching. A slit is provided for each pocket to create a cavity to retain a hot or cold pack, or other therapeutic aid within the pocket, as needed. The hook and loop fasteners attached to the edges may be 1¼ inches in width, and run across the length of the garment. The garment is stitched or otherwise manufactured in various sizes as per the requirements of the user. The garment in its closed position closely surrounds the upper torso of the wearer. The material of the garment and the elastic edges function to apply even pressure over the chest and rib cage areas of the patient while wearing the garment, and have a compression force ranging from 8 mmHg to 50 mmHg, with 15 mmHg to 30 mmHg being preferred.

In yet another embodiment of the present invention, a method of wearing a therapeutic garment having a pair of pockets to get relief from pain, swelling, and discomfort after chest surgery is disclosed. The method comprises the initial step of wrapping the therapeutic garment around the torso of the patient such that the pair of pockets cover the chest portion or areas that were subject to the surgery or treatment. Next, the therapeutic garment is closed in the front or back of the wearer using a closure mechanism such as, but not limited to, hook and loop fasteners, a zipper, buttons, clasps, snaps or other suitable fasteners. A cold pack, hot pack, dual hot-cold pack or other therapeutic device is then inserted into each of the pockets of the garment through the slits, and then the slits are closed to prevent the packs from falling out. The garment can be worn comfortably day and/or night, and does not interfere with the day to day activities of the wearer.

In a still further embodiment of the present invention, a compression garment is described and includes a web of material sized and configured to fit a human torso. The web of material is comprised of one or more of a Lycra, SMS, SMMS, SIVIMMS, a plurality of natural fibers, a plurality of synthetic fibers or combinations thereof. First and second pockets are applied to the web of material, with the first and second pockets having a size ranging from between 2 and 8 inches in length and between 2 and 6 inches in width. Therapeutic devices are inserted in each of the first and second pockets, and each of the first and second pockets are further comprised of a resealable closure. The web of material provides a compressive force to the chest area of the wearer of between 8 mmHg and 50 mmHg when applied and secured to the wearer's torso.

In a yet still further embodiment of the present invention, a post-surgical garment is described that comprises a web of material for providing a compression force to the chest area of the wearer. The web of material is comprised of a top edge and a bottom edge, with the top edge being secured around a patient and the bottom edge being open. The web of material is further comprised of first and second pockets disposed on a front surface, wherein each of the first and second pockets are sized and configured to receive a therapeutic aid. Each of the first and second pockets is further comprised of an openable slit to allow insertion of the therapeutic aid within the pocket, and the web of material provides a compression force ranging from between 8 mmHg to 50 mmHg.

The therapeutic treatment garment of the present invention is particularly advantageous because the material of the garment and the shape of the pockets are capable of flexibly changing to conform to the contours of the wearer's body. Further, the pockets are waterproof, and capable of retaining a cold pack, a hot pack or a dual hot-cold pack without dampening the wearer's skin. The dimensions of the pockets are made to fit, for example, a range of cold or hot packs, or a specifically sized cold or hot pack or other therapeutic aid. The garment is ready-to-wear and available in a large range of sizes.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of the therapeutic compression garment of the present invention in accordance with the disclosed architecture;

FIG. 2 illustrates a front perspective view of one potential embodiment of the right pocket stitched on the outer layer of the therapeutic compression garment of the present invention in accordance with the disclosed architecture;

FIG. 3 illustrates a front perspective view of an alternative potential embodiment of the therapeutic compression garment of the present invention in accordance with the disclosed architecture; and

FIG. 4 illustrates a perspective view of a user wearing one potential embodiment of the therapeutic compression garment of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there is a long felt need in the art for an effective treatment to combat post-operative inflammation, swelling in the chest area, pain, anxiety and the like. There is also a long felt need in the art for a therapeutic treatment garment that enables a wearer to apply heat or cold to an affected area in a hands free and secure manner, thereby enabling the wearer to engage in other tasks while receiving treatment. Additionally, there is a long felt need in the art for a therapeutic treatment garment that provides compression therapy to the affected area both during the application of the heat/cold and during such times that the same is not being applied, and that comprises waterproof pockets for securing and applying, for example, an ice pack to an affected area without dampening the skin of the wearer.

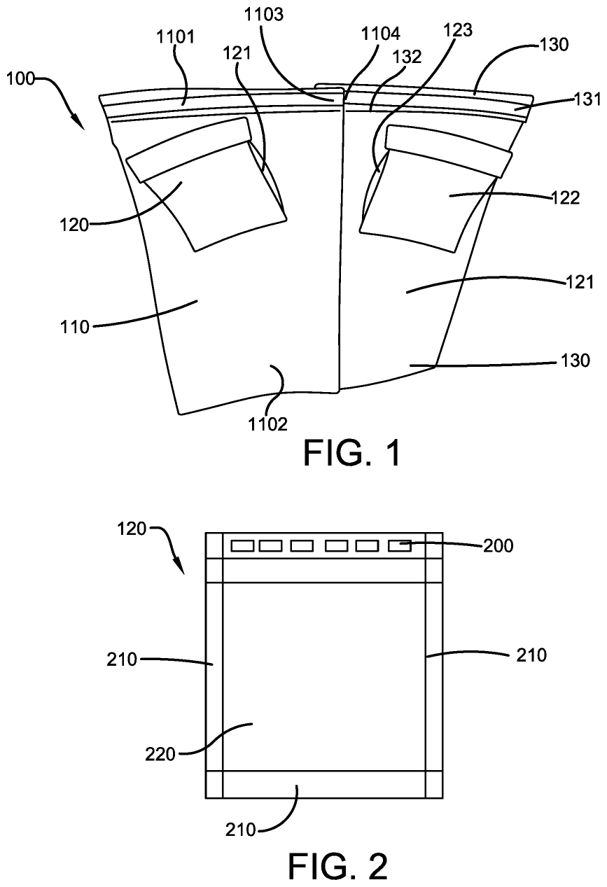

Referring initially to the drawings, FIG. 1 illustrates a front perspective view of one potential embodiment of the therapeutic compression garment 100 of the present invention in accordance with the disclosed architecture. More specifically, the therapeutic garment 100 is comprised of a web, band or tube-like post-surgical wrap 110 having a first longitudinal edge 1103, a second longitudinal edge 1104 which is opposite the first longitudinal edge 1104, a top edge 1101 and an opposite bottom edge 1102. In one embodiment, the first longitudinal edge 1103 has hook like fasteners along the length of the edge, and the second longitudinal edge 1104 has loop like fasteners along the length of the edge such that using the hook and loop fasteners allows the wrap 110 to be removably secured in the front or the back of the user 400.

When worn, the top edge 1101 of the wrap 110 is positioned at or near the top of the chest area of the user 400, and the bottom edge 1102 is open or free for relatively easy movement of the user 400. The therapeutic compression garment 100 is further comprised of a first pocket 120 and a second pocket 122 that are positioned on a front surface 121 of the post-surgical wrap 110, wherein the first or right pocket 120 covers the right chest area of the user 400 wearing the wrap garment 110, and the second or left pocket 122 covers the left chest area. Each of the pockets 120, 122 are preferably rectangular in shape and stitched on the outer or front surface 121 of the wrap 110 on three of the four sides of the pocket 120, 122, thereby leaving a slit or opening 121, 123. More specifically, the right pocket 120 is comprised of a slit or opening 121, and the left pocket 122 is comprised of a slit or opening 123. Each of the slits 121, 123 have a closure mechanism such as a magnetic fastener, hook and loop fastener, snap, zipper, button, clasp or other suitable closure for sealing or closing the pocket 120, 122.

The interior of the pockets 120,122 may receive a cold pack, a hot pack or a dual hot-cold pack, as per the preferences or needs of the user. In a preferred embodiment of the present invention, each of the pockets 120, 122 are stitched at an angle, such that no edge of the pockets 120, 122 is parallel to any of the top edge 1101, bottom edge 1102, or the longitudinal edges 1103, 1104. The position and shape of the pockets 120, 122 is meant to provide maximum coverage of the chest area by the cold pack, hot pack or the dual hot-cold pack kept in the interior of the pockets 120, 122.

As best shown in FIG. 1, a border 130 is preferably provided along the periphery of the wrap 110 to give a clean and comfortable finish to the wrap 110, such that it is securely worn by a user. The border 130 is created using stitching via thread and may include an elastic band 131 to help hold the wrap in place. The wrap garment 110 provides adequate relief from pain and discomfort, and is available in a large range of sizes with adjustability via a hook and loop fastener or front zipper along the longitudinal edges 1103, 1104. The wrap garment 110 is preferably manufactured in accordance with ASTM Test Method D 5034-90; Federal Test Method. Standard No. 191A, Method 5100 relating to strength and tear resistance. In addition, the wrap garment 110 should allow the passage of sterilant through the materials as well as anti-microbial solutions to protect against infection. The material is preferably waterproof and meets the hydrostatic head test—Federal Test Standard 191A, Method 5514 concerning the penetration of liquids. The material is made from one of SMS (Spunbond+Meltblown+Spunbond Nonwovens,), that is a combined nonwoven fabric which has two layers of spunbond material that have been combined with one layer of meltblown nonwovens inside, conforming them into a layered product, called a SMS nonwoven fabric. If the material is combined with two layer meltblown nonwoven inside, it is referred to as SMMS nonwoven fabric (spun-melt-melt-spun). In the same way, if the material is combined with three layer meltblown nonwoven inside, it is known as SMMMS nonwoven fabric (spun-melt-melt-melt-spun).

FIG. 2 illustrates a front perspective view of one potential embodiment of the right pocket 120 stitched on the outer layer of the therapeutic compression garment 100 of the present invention in accordance with the disclosed architecture. More specifically, the right pocket 120 is a generally rectangular shaped open-ended pocket having an interior cavity 220 for receipt of a cold pack, a hot pack or a dual hot-cold pack for applying cold or heat or therapeutic aid to the chest or other area. The right pocket 120 itself is stitched or sealed along three edges/borders 210, and has a slit opening having magnetic fasteners 200 to insert the therapeutic aid, such as the cold pack, hot pack or the dual hot-cold pack. The slit may also be reversibly sealable by means of opposite facing complementary hook and loop fastener strips. Nonetheless, it should be appreciated that any suitable fastener, such as a zipper, male and female connectors, clips, buttons, clasps, etc. can reversibly seal the slit of the right pocket 120. Further, while the right pocket 120 is shown as a rectangle, it should be understood that the pocket may be of any geometric shape in order to accommodate different configurations and apply different treatment regimes. It should also be appreciated that the details of the left pocket 122 can be the same as of the right pocket 120 described in this embodiment. If the pockets 120, 122 are not stitched in place, they may be removable, such as through the use of a removable or repositionable adhesive so that the garment does not need to have two pockets, or any pockets.

As previously stated, a cold pack, such as a freezer pack or crushed ice secured inside a zip-lock bag can be used in the pockets 120, 122. A hot pack could be, for example, heatable beads, a gel pack or the like secured inside a bag. In one embodiment, the pockets 120, 122 are made up of the same material as the wrap 110 and, in another embodiment, the material of the pockets 120, 122 and the wrap 110 may be different. The pockets 120, 122 are preferably waterproof and non-insulating in nature, such that the heat or cold from the packs when placed in the pockets 120, 122 reduces swelling, pain and discomfort. The size of the pockets 120, 122 may range from between 2 and 8 inches in length, and between 2 and 6 inches in width.

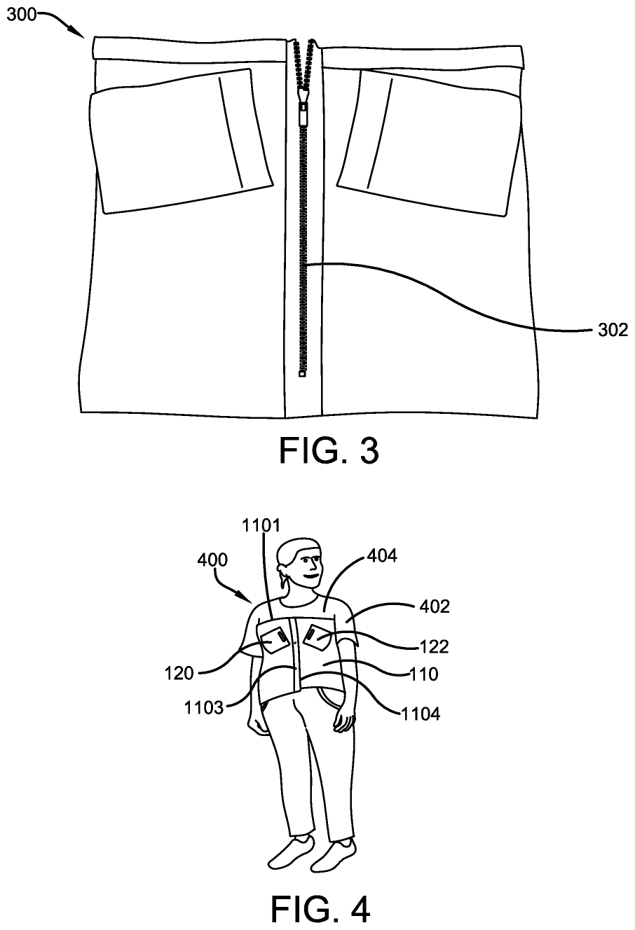

FIG. 3 illustrates a front perspective view of an alternative potential embodiment of the therapeutic compression garment 300 of the present invention in accordance with the disclosed architecture. More specifically, the therapeutic compression garment 300 is comprised of a zip fastener 302 on its longitudinal edges 1103, 1104 to secure the edges together when the garment 300 is worn by the user. Each longitudinal edge 1103, 1104 has rows of protruding teeth to secure the edges together. The zip fastener 302 allows the garment 300 to more easily conform to the shape of the torso of the user.

FIG. 4 illustrates a perspective view of a user 400 wearing one potential embodiment of the therapeutic compression garment 100 of the present invention in accordance with the disclosed architecture. More specifically, the user 400 is wearing the wrap garment 110 over a t-shirt 402. In other embodiments, the user 400 can wear the garment 100 under their clothes. As shown, the right pocket 120 covers the right chest area 404 and the left pocket 122 covers the left chest area of the user 400. The pain alleviating or therapeutic packs are inserted into the pockets 120, 122 to soothe the chest area 404 or other areas, such as the back of the user 400. The material of the wrap 110 is such that the garment 100 conforms and secures comfortably around the torso of the user 400. The top edge 1101 rests above the chest area 404 to ensure that the complete chest of the user 400 is covered by the garment 100, and may include an elastic band 131. When the garment 100 is worn by the user 400, the longitudinal edges 1103, 1104 are secured in the front or back of the user 400, depending on the area being treated using a fastening means, as described in FIG. 1. The magnetic or other fastener present on the slits of the pockets 120, 122 is closed once the garment 110 is worn by the user 400 with the therapeutic packs present within the pockets 120, 122. As previously stated, the garment 100 may be comprised of any suitable material, such as, but not limited to, stretchable cotton, Lycra®, SMS, SMMS, SMMMS, natural and synthetic fibers as well as combinations of woven and non-woven materials.

In one embodiment, the therapeutic compression garment 100 may have adjustable straps (not shown) for required comfort and support, or one or more internal pockets to hold, for example, drains. The internal pockets may further comprise special fasteners to keep the drains and drainage tubes in place. The garment 100 of the present invention can be worn in all types of weather, and is relatively lightweight. The material is soft, breathable, and has wrinkle resistant properties. The garment 100 is ideal for post-surgery recovery and rehab for providing relief from pain, swelling, muscle spasms, and discomfort while maintaining privacy, comfort and style; and is suited to both single and double mastectomies, heart and kidney surgery.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “therapeutic garment”, “compression garment”, “therapeutic compression garment”, and “post-surgical wrap” are interchangeable and refer to the therapeutic garment 100 of the present invention.

Notwithstanding the forgoing, the therapeutic compression garment 100 of the present invention can be of any suitable size and configuration as is known in the art without affecting the overall concept of the invention, provided that it accomplishes the above stated objectives. One of ordinary skill in the art will appreciate that the size, configuration and material of the therapeutic garment 100 as shown in the FIGS. are for illustrative purposes only, and that many other sizes of the therapeutic garment 100 are well within the scope of the present disclosure. Although the dimensions of the therapeutic compression garment 100 are important design parameters for user convenience, the therapeutic compression garment 100 may be of any size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A therapeutic garment comprising;
a band of material sized and configured to fit around a torso of a patient and comprising a front surface; and
at least one pocket secured to the front surface of the band of material, wherein the at least one pocket is sized and configured to receive a therapeutic device, and further wherein the at least one pocket is comprised of a resealable closure and is waterproof.

2. The therapeutic garment as recited in claim 1, wherein the band of material is manufactured in accordance with ASTM Test Method D 5034-90; Federal Test method Standard No. 191A, Method 5100.

3. The therapeutic garment as recited in claim 1, wherein the at least one pocket satisfies the hydrostatic head test—Federal Test Standard 191A, Method 5514

4. The therapeutic garment as recited in claim 1 further comprising a second pocket disposed on the front surface of the band of material.

5. The therapeutic garment as recited in claim 1, wherein the band of material is comprised of one of a Lycra, a SMS, a SMMS, a SMMMS, or a combination thereof.

6. The therapeutic garment as recited in claim 1, wherein the at least one pocket has a length of between 2 and 8 inches, and a width of between 2 and 6 inches.

7. The therapeutic garment as recited in claim 1, wherein the band of material provides a compression force ranging from 8 mmHg to 50 mmHg.

8. The therapeutic garment as recited in claim 1, wherein the band of material provides a compression force of between 15 mmHg to 30 mmHg.

9. The therapeutic garment as recited in claim 1, wherein the resealable closure is one of a hook and loop fastener, a magnet, a snap, a clap, a button, a zipper, or a combination thereof.

10. The therapeutic garment as recited in claim 1, wherein the band of material is comprised of a plurality of natural fibers, a plurality of synthetic fibers, or a combination thereof.

11. A post-surgical garment comprising:
a web of material for providing a compression force, wherein the web of material comprises a top edge and a bottom edge, and further wherein the top edge being secured around a patient and the bottom edge being open;
the web of material having a first pocket and a second pocket each disposed on a front surface of the web of material, and each of the first and second pockets sized and configured to receive a therapeutic aid;
each of the first and second pockets having an openable slit to allow insertion of the therapeutic aid within the first and second pockets; and
the web of material providing a compression force ranging from between 8 mmHg to 50 mmHg.

12. The post-surgical garment as recited in claim 11, wherein each of the first and second pockets are waterproof.

13. The post-surgical garment as recited in claim 11, wherein the therapeutic aid is one of a hot pack or a cold pack.

14. The post-surgical garment as recited in claim 11, wherein the openable slit includes a closure.

15. The post-surgical garment as recited in claim 14, wherein the closure is one of a hook and loop fastener, a magnet, a snap, a clap, a button, a zipper or the combination thereof.

16. The post-surgical garment as recited in claim 11, wherein web of material is comprised of one of a Lycra, a SMS, a SMMS, a SMMMS, a plurality of natural fibers, a plurality of synthetic fibers or a combination thereof.

17. A compression garment system comprising:
a web of material sized and configured to fit a human torso, wherein the web of material is comprised of at least one of a Lycra, a SMS, a SMMS, a SMMMS, a plurality of natural fibers, a plurality of synthetic fibers or a combination thereof;
a first pocket and a second pocket each applied to the web of material, wherein each of the first and second pockets have a length ranging from between 2 and 8 inches and a width ranging from between 2 and 6 inches;
a therapeutic device inserted in each of the first and second pockets, wherein each of the first and second pockets are comprised of a resealable closure; and
the web of material providing a compressive force of between 8 mmHg and 50 mmHg when applied and secured to the human torso.

18. The compression garment system as recited in claim 17, wherein each of the first and second pockets are waterproof.

19. The compression garment system as recited in claim 17, wherein the web of material is comprised of a top edge and a bottom edge, wherein the top edge is secured around a patient and the bottom edge is open.

20. The compression garment system as recited in claim 19, wherein the top edge includes an elastic material.
